(19) United States
(12) Patent Application Publication
(10) Pub. No.: US 2005/0238742 A1
(43) Pub. Date: Oct. 27, 2005

(54) Extraction method for effectively obtaining amygdalin from persicae semen or armenicae semen

(76) Inventors: Seon-Pyo Hong, Seoul (KR); Jae-Young Kim, Seoul (KR)

Correspondence Address: KIRK HAHN, 14431 HOLT AVE, SANTA ANA, CA, 92705, US

(21) Appl. No.: 10/524,969

(22) PCT Filed: Jul. 23, 2003
(86) PCT No.: PCT/KR03/01477

§ 371 Date: Feb. 19, 2005

(51) Int. Cl. A61K 35/78

(52) U.S. Cl. 424/776

(57) ABSTRACT

The present invention relates to the useful method for the optimum extraction condition of amygdalin from Persicae Semen or Armenicae Semen. Persicae Semen is the herb medicine that contains amygdalin as a major ingredient. It has been generally used as a lubricant or an anti-stasis agent in traditional oriental medicine. Amygdalin in water is decomposed into benzaldehyde, HCN, and glucose by emulsin, a hydrolysis enzyme in Persicae Semen. A useful and practical method for the optimum extraction condition of amygdalin without enzymatic hydrolysis is required. We can be able to provide the optimum extraction condition for maximum extraction yield of amygdalin from Persicae Semen and extrate amygdalin, not only controlling with the cutting size of Persicae Semen but also using the water which has the temperature of boiling point or the acid containing aqueous solution in order not to be decomposed by emulsin, a hydrolysis enzyme in Persicae Semen.

### CROSS REFERENCE TO RELATED APPLICATION

This application is a continuation patent application of PCT Patent Application No. PCT/KR2003/001477, which was filed on Jul. 23, 2003, designating the United States of America, now abandoned.

### FIELD OF THE INVENTION

The present invention relates to an extraction method for obtaining amygdalin from Persicae Semen or Armenicae Semen effectively.

### BACKGROUND OF THE INVENTION

The present invention relates to an optimum extraction method for obtaining maximum extraction yield of amygdalin having anti-cancer activity.

Cancer is a subject to overcome with first priority with a view to extending human life span. Currently, the ratio of cancer occurrence has been increasing by about 5% year by year because of the increase ratio of old people and environmental deterioration. The number of dead people taken with cancer disease was 6,000,000 in 1997, which falls under 12% of total world-wide mortality. 100,000 numbers of patients taken with cancer have been newly found and 50,000 number of plasmsatients died every year. The increasing rate of cancer patient is 10%, the number of cancer patient is 120,000 and cancers occur in the order of stomach cancer (21%), liver cancer (12%), lung cancer (11%) in men, and cervical cancer (20%), stomach cancer (16%), breast cancer (13%) in women.

Most of anti-cancer agent shows anti-cancer activity caused by the inhibition of nucleic acid synthesis, i.e., it intervenes various metabolism pathway. However, those anti-cancer agent acts on not only cancer cells selectively but also normal cells, which gives rise to various side effect such as bone marrow malfunction, gastrointestinal disorder and alopecia by causing damage to organic cell of which cell division is active. Anti-cancer agent can be classified into six categories according to their biochemical mechanism.

Anti-cancer agent can be classified with a chemotherapy agent and a biological therapy agent: A chemotherapy agent is highly reactive and is synthetic chemical substance such as an alkylating agent to transform DNA structure or to cleave DNA sequence; An anti-metabolite to inhibit metabolic pathway in multiplying cancer cells; An antibiotic having anti-cancer activity isolated from various microorganism, natural product and hormonal drug. A biological therapy agent can be classified with several agents: An immuno-therapy agent to remove caner cells by way of enhancing immune response of patients taken with cancer using by therapeutic vaccine, monoclonal antibody and cytokine; A mitotic inhibitor, for example, vinca alkaloid, to obstruct the cell differentiation in metaphase of mitosis as a drug specific to cell division cycle; A gene therapy agent which is administered to the patient having cancer disease, caused by gene deficiency or gene mutation, for treating cancer by the mechanism of controlling abnormal gene and producing therapeutic protein in cell or tissue; An anti-cancer agent containing antisense, nucleic acid having oncogene-inhibitory activity with selectively binding to oncogene; and an angiogenesis-inhibiting agent.

Anti-cancer agent acts to cancer cells more selectively with low toxicity to normal cells according to the difference of sensitivity about drug in normal and cancer cells, however, it shows the side effects because normal cells is also damaged more or less.

An anti-cancer agent acts on cancer cells more selectively with low toxicity to normal cells according to the difference of sensitivity about drug in normal anc cancer cells, however, it shows the side effects because normal cells is also damaged more or less. An anti-cancer agent acts not only on quickly-dividing cancer cells but also on normal cells such as bone marrow, gastrointestinal tract, hair root cells because it can operate wherever division of cell occurs fast. Therefore, bone marrow, gastrointestinal tract and hair root cells are affected by anti-cancer agent and various side effects such as the decrease of leukanemia, nausea, vomiting, diarrhea, anorexia and alopecia may appear during anti-cancer treatment.

Although anti-cancer agent has been considerably developed for cancer treatment, the satisfactory anti-cancer agent without side effect, drug tolerance or recurrence of disease has been not yet developed. Therefore, we attempt to develop the safe and effective anti-cancer agent using traditional oriental medicine.

Armenicae semen, seed of Prunus armeniace Linne var. ansu Maximowicz belonged to Rosaceae or other same genus, is the traditional oriental medicine for treating asthma, dyspnea, edema and so on. It has been reported that Armenicae semen contains about 3% of amygdalin group compounds, 50% of fatty oils (armenicae semen oils) and various amino acid. Amygdalin, i.e., vitamin B17, is hydrolyzed by β-glycosidase such as amygdalase and prunase among Armenicae semen to produce prunasin and benzaldehyde and further, degraded to form benzaldehyde and HCN. (B. S. Chung and M. K. Shin; HyangyakDaesajeon, Youngrim Co., pp 625-626, 1998)

Also, Persicae Semen, seed of Prunus persica Batsch, Prunus persica var. davidiana Maximowicz belonged to Rosaceae has been used as a lubricant or an anti-platelet agglutination agent long years ago in Asian countries. It has been reported that Persicae semen contains about 3.6% of amygdalin group compounds, 0.4% of essential oils, 45% of fatty oils such as olefin type glycerin, linolenic glycerin and other trace components, for example, palmitic acid, stearic acid, choline, acetylcholine, emulsin and so on (B. S. Chung and M. K. Shin; HyangyakDaesajeon, Youngrim Co., pp 632, 1998). Amygdalin, a cyan hydrogenated glycoside, as a main ingredient of Persicae semen and Armenicae semen, is hydrolyzed by emulsin in the presence of water to form mandelonitrile and further decomposed into benzaldehyde, HCN and glucose. Recently, it has been reported that amygdalin can kill cancer cells selectively at specific tumor site without systemic side effect occurred in case of conventional chemical substance (Syrigos, K. N., Rowlinson-Busza, G. and Epenetos, A. A., International Journal of Cancer, 78, pp 712-719, 1998).

We attempted to develop anti-tumor agent using oriental medicine containing Persicae semen or Armenicae semen having several advantages such as low cost without side effect.

Generally, Persicae semen is used by being extracted in the form of powdered seed remaining its husk and Armenicae semen is used by being extracted in the form of powdered seed removing its husk. However, in case that Persicae semen and Armenicae semen are extracted by above mrthod, amygdalin being contained in Persicae semen and Armenicae semen is ready to be decomposed by emulsin, and therefore, it has been reported that the content of amygdalin is only about 0.08% from husked Persicae semen and about 0.24% from unhusked Persicae semen in the literature (Hong, S. P. et al., Archives of Pharamcal Reserch, 25(4), 2002).

Therefore, since the pharmacolgical activity of the seed extract containig few contant of amygdalin prepared by conventional procedure is not enough to use as a anti-cancer agent and further, about the half amount of the amygdalin is converted to neoamydalin which has no anti-cancer activity, more efficient preparation method has been needed till now.

Prevent inventors have endeavored to deveolope to find efficient extraction method to obtain high yield of amygdalin without hydrolysis of emulsin. Finally, we found out most effcient condition to accomplish high yield amygdalin and completed the prevent invention.

### SUMMARY OF THE INVENTION

The present invention provides optimum extraction method for obtaining maximum extraction yield of amygdalin from Persicae semen or Armenicae semen.

### BRIEF DESCRIPTION OF THE DRAWINGS

The above and other objects, features and other advantages of the present invention will be more clearly understood from the following detailed description taken in conjunction with the accompanying drawings, in which;

FIG. 1 shows a chromatogram of amygdalin from Persicae semen by reversed-phase seperation with a 25% methanol as a mobile phase after extraction with water;

FIG. 2 shows extraction yield of amygdalin according to cutting size of unhusked Persicae Semen with methanol;

FIG. 3 shows extraction yield of amygdalin according to cutting size of husked Persicae Semen with methanol;

FIG. 4 shows extraction yield of amygdalin according to cutting size of unhusked Persicae Semen with water;

FIG. 5 shows extraction yield of amygdalin according to cutting size of husked Persicae Semen with water;

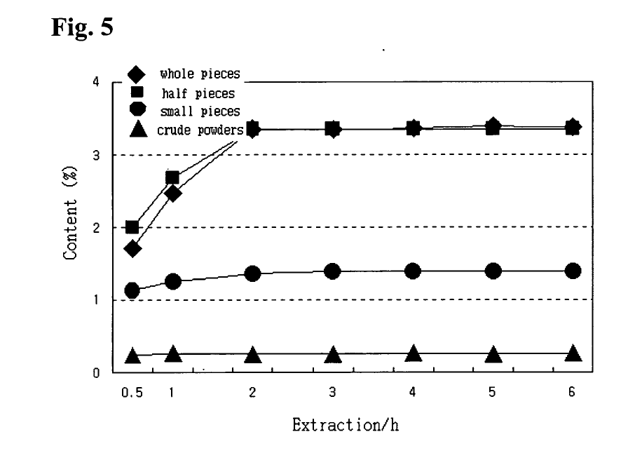

FIG. 6 shows effects of emulsin on extraction of amygdalin with water;

FIG. 7 shows extraction yield of amygdalin according to cutting size of unhusked Armenicae Semen with methanol;

FIG. 8 shows extraction yield of amygdalin according to cutting size of husked Armenicae Semen with methanol;

FIG. 9 shows extraction yield of amygdalin according to cutting size of unhusked Armenicae Semen with water;

FIG. 10 shows extraction yield of amygdalin according to cutting size of husked Armenicae Semen with water;

FIG. 11 shows extraction yield of amygdalin according to cutting size of husked Armenicae Semen with boiling water;

FIG. 12 shows extraction yield of amygdalin from whole pieces of husked Armenicae Semen with boiling water comprising 0.1% of citric acid.

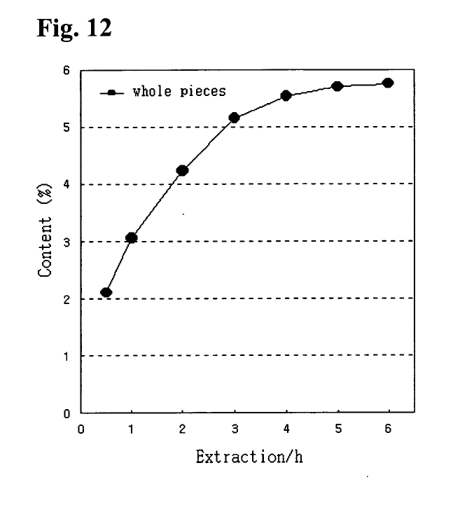

### DETAILED DESCRIPTION OF THE INVENTION

Accordingly, it is an object of the present invention to provide a method characterized in minimizing the excised surface of Persicae semen or Armenicae semen in extracting Persicae semen or Armenicae semen with extraction solvent following acid pre-treatment.

It is preferable that the husk of above described Persicae semen or Armenicae semen is used as removed.

Above described extraction solvent comprises at least one selected from the group consisting of water, methanol, butanol or the mixture thereof, preferably hot water.

Also, preferably, the acid pre-treatment in above described method is performed with at least one acid selected from the group consisting of citric acid, acetic acid, ascorbic acid or the mixture thereof, more preferably, 0.05˜0.5% citric acid, most preferably, water solution containing 0.1% citric acid at higher temperature above its boiling point.

Also, it is another object of the present invention to provide an extraction method characterized in minimizing the contact area between amygdalin and emulsin, preferably cutting into half piece, more preferably whole piece.

Additionally, it is an object of the present invention to provide the extraction method for isolating amygdalin from Persicae semen characterized by using crude powder for providing maximum surface.

An inventive extraction method of amygdalin from Persicae Semen or Armenicae Semen may be prepared in accordance with the following preferred embodiment.

Hereinafter, the present invention is described in detail.

An inventive extraction method of amygdalin from Persicae Semen or Armenicae Semen can be prepared by following procedures: Persicae Semen or Armenicae Semen is dried, cut in crude powder, small pieces, half pieces and whole pieces, crushed and mixed with 5 to 20-fold, preferably, approximately 10 to 15-fold volume of distilled water, lower alcohols such as methanol, ethanol, butanol and the like, or the mixtures thereof, preferably water at 100° C. or methanol at 64.5° C. of boiling point, for the period ranging from 30 mins to 6 hours with extraction method selected from one among the extraction with hot water, cold water, reflux extraction, or ultra-sonication extraction with 1 to 5 times, preferably 2 to 3 times, consecutively; the residue is filtered to obtain the supernatant to be concentrated with rotary evaporator then dried by vacuum freeze-drying, hot air-drying or spray drying to obtain water or methanol soluble extract of Persicae Semen or Armenicae Semen; and further, the water or methanol extract prepared by above step, is suspended in water; and then is mixed with 1 to 100-fold, preferably, 1 to 5-fold volume of non polar solvent such as ethyl acetate, chloroform, hexane and the like and fractioned 1 to 10 times, preferably, 2 to 5 times to remove non-polar solvent soluble extract and obtain remaining water soluble layer; the water soluble layer is subjected to HPLC to obtain amygdalin abundant fraction.

Hereinafter, an inventive extraction method of amygdalin from Persicae semen or Armenicae semen can be explained by following procedures;

For example, at first step, Persicae semen is cut into crude powder, small piece, half piece or whole piece, extracted with polar solvent such as water, methanol, ethanol, butanol and the like, and the residue is filtered, the supernatant is concentrated with a rotary evaporator to obtain the soluble extract of polar solvent.

At second step, the extract prepared by above step is suspended in water, and then is separated with non-polar solvent such as ethyl acetate, chloroform, hexane and the like; and the non-polar soluble extract is discarded; and finally, remaining polar solvent soluble extract is used as a test sample of high performance liquid chromatography (HPLC).

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Reference Example, Examples and Experimental Examples are intended to further illustrate the present invention without limiting its scope.

### Examples 1

### Preparation of Methanol Soluble Extract of Unhusked Persicae Semen

Dried Persicae Semen (Herb market, Dae-gu) was prepared in the form of crude powders, small pieces, half pieces and whole pieces. Each 2 g of sample was extracted under reflux with 50 mL of methanol for 0.5, 1, 2, 3, 4, 5 and 6 hours, respectively. Each methanol extract was filtered with a filter paper(Whatman Co.) to remove the debris and condensed under reduced pressure. After suspending in 50 mL of distilled water, 50 mL of n-hexane was added thereto in a separatory funnel and divided into n-hexane insoluble layer (lower layer) and n-hexane soluble layer (upper layer). And then n-hexane insoluble layer was collected. Lower layer (aqueous layer) was treated with equivalent volume of n-hexane over three times to remove nonpolar substances according to above-described method. Remaining aqueous layers were concentrated and dried to use as a sample in the following experiments.

### Example 2

### Preparation of Water Soluble Extract of Unhusked Persicae Semen

Extract sample was obtained by identical method described in Example 1 excepting by using water as an extracting solvent.

### Examples 3

### Preparation of Methanol Soluble Extract of Husked Persicae Semen

Dried and husked Persicae Semen was used in extraction. And extract sample was obtained by identical method described in Example 1 excepting by using methanol as an extracting solvent.

### Examples 4

### Preparation of Water Soluble Extract of Husked Persicae Semen

Dried and husked Persicae Semen (Herb market, Dae-gu) was used in the extraction. And extract sample was obtained by identical method described in Example 1 excepting by using water as an extracting solvent.

### Examples 5

### Preparation of Methanol Soluble Extract of Unhusked Armenicae Semen

Dried Armenicae Semen (Chungbuk, Moseingdang Oriental medical store) was selected carefully. And an extract sample was obtained by identical method described in Example 1 excepting by using methanol as an extracting solvent.

### Examples 6

### Preparation of Methanol Soluble Extract of Husked Armenicae Semen

Dried Armenicae Semen (Chungbuk, Moseingdang Oriental medical store) was husked. And an extract sample was obtained by identical method described in Example 1 excepting by using methanol as an extracting solvent.

### Examples 7

### Preparation of Water Soluble Extract of Unhusked Armenicae Semen

Dried Armenicae Semen (Chungbuk, Moseingdang Oriental medical store) selected carefully, was extracted under reflux with water as an extracting solvent by identical method described in Example 1 to obtain the inventive extract.

### Examples 8

### Preparation of Water Soluble Extract of Husked Armenicae Semen

Dried and husked Armenicae Semen (Chungbuk, Moseingdang Oriental medical store), was extracted with water as an extracting solvent under reflux by the identical method described in Example 1 to obtain the inventive extract.

### Examples 9

### Preparation of boiling Water Soluble Extract of Husked Armenicae Semen

Dried and husked Armenicae Semen (Herb market, Dae-gu), was prepared in the form of crude powders, small pieces, half pieces and whole pieces. 2 g of each sample was mixed with 50 mL of boiling water in 100 mL volume of beaker and the mixture was extracted under reflux by the identical method described in Example 1 for 0.5, 1, 2, 3, 4, 5 and 6 hours to obtain the inventive extract.

### Examples 10

### Preparation of Boiling Water Soluble Extract Comprising 0.1% Citric Acid of Husked Armenicae Semen

Whole piece of dried and husked Armenicae Semen (Herb market, Dae-gu) was boiled with 50 mL of 0.1% citric acid in 100 mL beaker. The 2 g of whole piece of Persicae Semen was extracted under reflux by identical method described in Example 1 for 0.5, 1, 2, 3, 4, 5 and 6 hours to obtain the inventive extract.

### Reference Example 1

### High Performance Liquid Chromatography (HPLC) Apparatus

The HPLC was detected at 214 nm using a M930 pump (Young Lin Co., Kyunggi, Korea) with M720 UV detector. The column was a Capcell Pak C18, Type UG120(250 mm×4.6 mm, 5 μm, Shiseido, Japan) maintained at 35° C. in a CTS30 column oven(Young Lin, Kyunggi, Korea). Mobile phase was 25% methanol-water (methanol:water=25:75) at 1 mL/min of flow rate.

### Experimental Example 1

### High Performance Liquid Chromatography Calibration

To standardize high performance liquid chromatography, a calibration was carried out and thereby its linearity was confirmed. Analytic amygdalin (Tokyo Hwasung, Japan) was used as a standard solution and distilled water was refined with Pure system (Pure power III, Taiwan).

A Capcell Pak C18 was set in HPLC column holder, the flow rate was 1 mL/min and UV detector was set at 214 nm. The column was washed with 25% methanol for 1 hour. In the case of manual injection, the 10 μl of standard solution containing 30 μg/ml, 60 μg/ml, 90 μg/ml, 120 μg/ml, 150 μg/ml and 300 μg/ml of amygdalin sample prepared in Example 1, respectively, was injected with syringe. HPLC system was operated at room temperature.

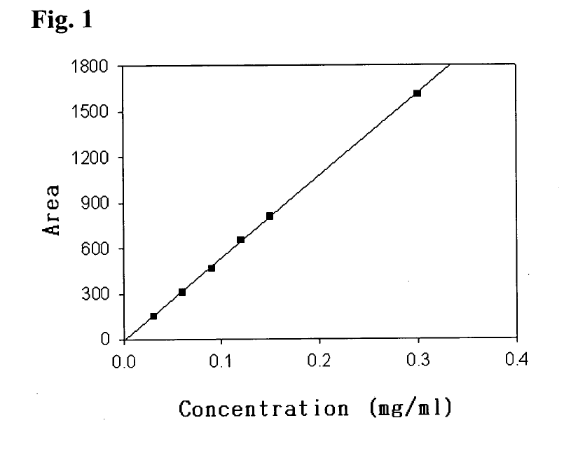

As a result of experiment, the peak of amygdalin was completely separated in our method without any pretreatment and the calibration curve between peak area and the concentration of standard amygdalin showed excellent linearity (r²=0.9996)(FIG. 1).

### Experimental Example 2

### Amygdalin Extraction Yield Calibration of Methanol Extract of Natural Form Persicae Semen

Each methanol extract of the crude powders, small pieces, half pieces and whole pieces of unhusked Persicae Semen prepared in Example 1 was subjected to HPLC analysis according to a pretreatment of above described Example 1 and Experimental Example 1.

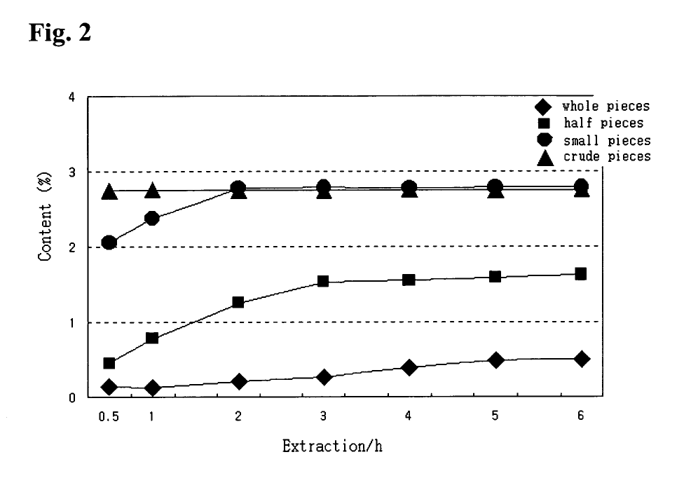

As a result of experiment, the content of amygdalin in methanol extract was 2.8% from crude powders, 2.8% from small pieces, 1.6% from half pieces and 0.5% from whole pieces. The extraction yields from crude powders and from small pieces were almost the same, but the time to take for extraction was 0.5 hour from crude powders and 2 hours from small pieces. This result indicated that the smaller size of natural form Persicae Semen showed the superior extraction yield and ratio (FIG. 2).

### Experimental Example 3

### Amygdalin Extraction Yield Calibration of Methanol Extract of Husked Persicae Semen

Each methanol extract of the crude powders, small pieces, half pieces and whole pieces of husked Persicae Semen prepared in Example 3 was subjected to HPLC analysis according to a pretreatment of above described Example 3 and Experimental Example 1.

As a result of experiment, the content of amygdalin in methanol extract was 3.2% from crude powders, 3.2% from small pieces, 2.2% from half pieces and 1.5% from whole pieces. Compared with unhusked Persicae Semen, the patterns of extraction yield and extraction ratio according to cutting sizes were similar, but the extraction yield itself was generally much increased. This result reconfirmed that the smaller size of husked Persicae Semen showed the superior extraction yield and ratio same as that of methanol extract of natural form Persicae Semen above described in Experiment Example 2.

### Experimental Example 4

### Amygdalin Extraction Yield Calibration of Unhusked Persicae Semen Extract with Water

Each water extract of the crude powders, small pieces, half pieces and whole pieces from unhusked Persicae Semen prepared in Example 2 was subjected to HPLC analysis according to a pre-treatment of above described Example 2 and Experimental Example 1.

As a result of experiment, the amygdalin yield of water extract of unhusked Persicae Semen was different from the that of methanol extract and the content of amygdalin in water extract was 0.1% from crude powders, 1.4% from small pieces, 3.5% from half pieces and 2.4% from whole pieces. And it had a shortcoming taken 4 hours for complete extraction.

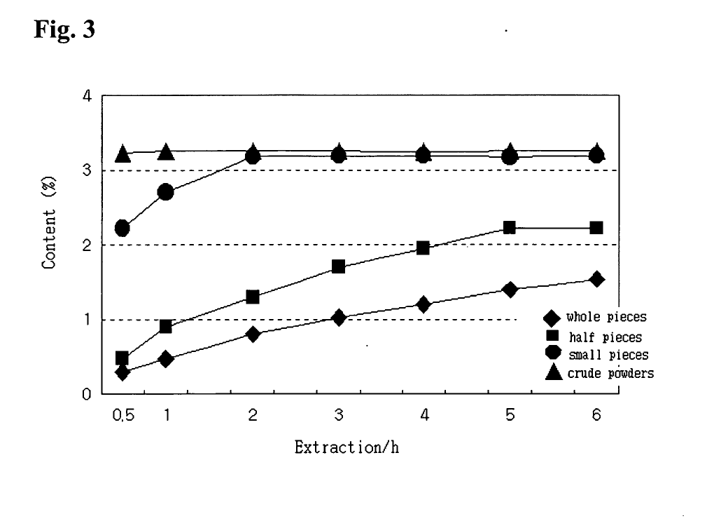

It was found that the amygdalin from crude powders of Persicae Semen was mostly degraded by the effect of emulsin, a kind of hydrolase in the water extract, but emulsin in methanol extract did not work. Actually amygdalin is decomposed into two molecules of glucose, a molecule of HCN and a molecule of benzaldehyde by emulsin, also β-glucosides such as salisin, arbutin, cellobiose and the like are also decomposed by emulsin. But because the amygdalin from half pieces of Persicae Semen was not degraded by emulsin, it indicates that the effect of emulsin is decreased with the increase of the cutting size (FIG. 3).

### Experimental Example 5

### Amygdalin Extraction Yield Calibration of Husked Persicae Semen Extract with Water

Each water extract of the crude powders, small pieces, half pieces and whole pieces from husked Persicae Semen prepared in Example 4 was subjected to HPLC analysis according to a pre-treatment of above described Example 4 and Experimental Example 1.

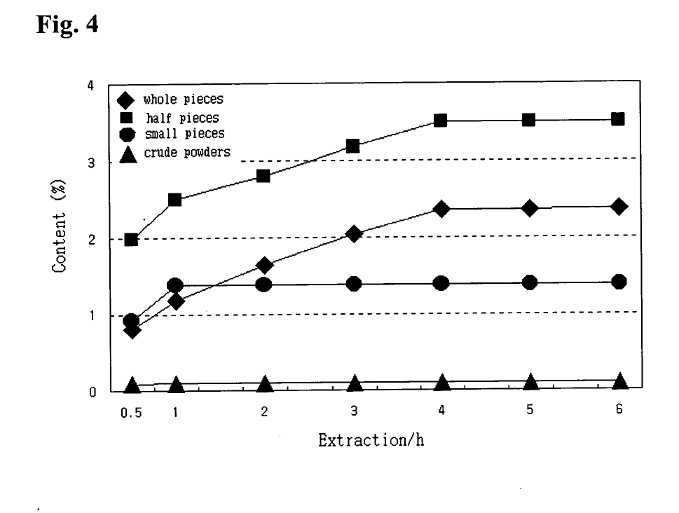

As a result of experiment, the content of amygdalin of husked Persicae Semen was 0.3% from crude powders, 1.4% from small pieces, and 3.5% from half pieces and whole pieces, respectively. The amygdalin isolated from half pieces and whole pieces of Persicae Semen was completely extracted at 2 hours without interference of emulsin. Therefore, we could find that emulsin had no effect on the decomposition of amygdalin in the use of seed in the size larger than the half (FIG. 4).

### Experimental Example 6

### Effects of Emulsin on the Extraction of Amygdalin

To find out the major existing part of emulsin and the mechanism of emulsin, the experiment about effects on the emulsin on the extraction yield of amygdalin was carried.

The each powder of unhusked Persicae Semen, husked Persicae Semen and inner shell-eliminated Persicae Semen was prepared and extracted with 50 mL of water under reflux at 100° C. for 2 hours. Each supernatant was filtered with filter paper to remove the debris, and then 50 mL of n-hexane was added thereto in separatory funnel and divided into n-hexane insoluble layer (lower layer) and n-hexane soluble layer (upper layer). And then n-hexane insoluble layer was collected.

Non-polar substances were removed from n-hexane insoluble layer by treating with n-hexane over three times. Remaining aqueous layers were used as a sample of HPLC analysis, HPLC analysis was followed by above described in Experimental Example 1.

As a result of experiment, content of amygdalin was about 0.1% from water extract of unhusked Persicae Semen, 0.3% from husked Persicae Semen, and 3.2% from powder form of inner shell-eliminated Persicae Semen.

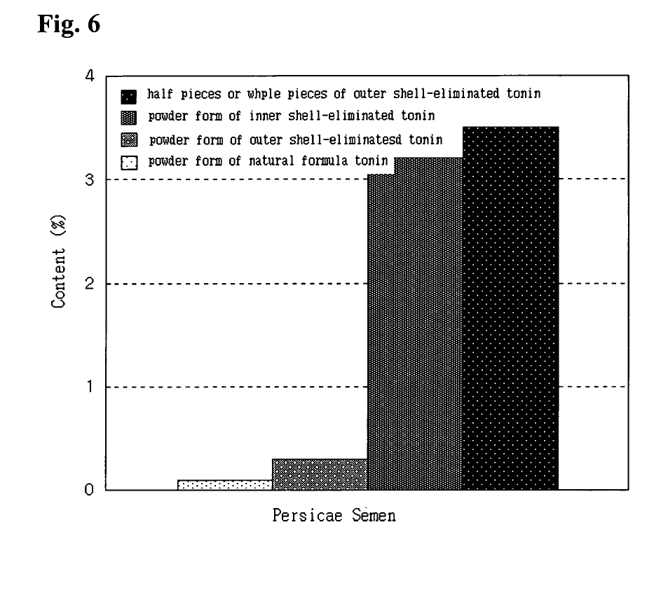

In this study, we could found out that emulsin is mainly contained in inner shell part. Emulsin was extracted and inactivated by boiling water before the extraction of amygdalin so that amygdalin could be obtained up to almost 100% without interference of emulsin. However, it was confirmed that amygdalin was almost decomposed by emulsin in using crude powders of unhusked or husked Persicae Semen, because emulsin was adjacent to amygdalin and hydrolysis of amygdalin occurred faster than the inactivation of emulsin by boiling water (FIG. 6).

In the result, the extract yield of amygdalin from crude powder methanol extracts showed the highest extraction efficiency among other methanol extracts because the powder form had the largest surface area and therefore, the emulsin could not work at all. On the other hand, the extraction yield of amygdalin from crude powder water extract showed the lowest extract efficiency because amygdalin was almost decomposed by adjacent emulsin. If the sizes of Persicae Semen become larger, amygdalin is not decomposed to improve the extraction yield because the contact area between emulsin and amygdalin loses.

Therefore we confirmed that the ideal condition of extraction is that extract is prepared from husked Persicae Semen having below half size, which gives the maximum yield, short extraction time and no effect of emulsin on amygdalin.

According to above results, we found out the optimum extraction condition of amygdalin from Persicae Semen or Armenicae Semen with each extraction solvent without interference of emulsin and thereby it was confirmed that inventive preparation method can provide the mass production and high yield of amygdalin as an anti-cancer reagent possible.

### Experimental Example 7

### Amygdalin Extraction Yield Calibration of Unhusked Armenicae Semen Extract with Methanol

Each methanol extract of the crude powders, small pieces, half pieces and whole pieces from unhusked Armenicae Semen prepared in Example 5 was subjected to HPLC analysis according to a pretreatment of above described Example 5 and Experimental Example 1.

As a result of the experiment, the amygdalin content of unhusked Armenicae Semen was 5.2% from crude powders, 5.1% from small pieces, 1.7% from half pieces and 1.2% from whole pieces. The amygdalin content of crude powders was almost same as that of small pieces, but complete extraction time was different each other, i.e., 4 hours for crude powders and 5 hours for small pieces of Armenicae Semen.

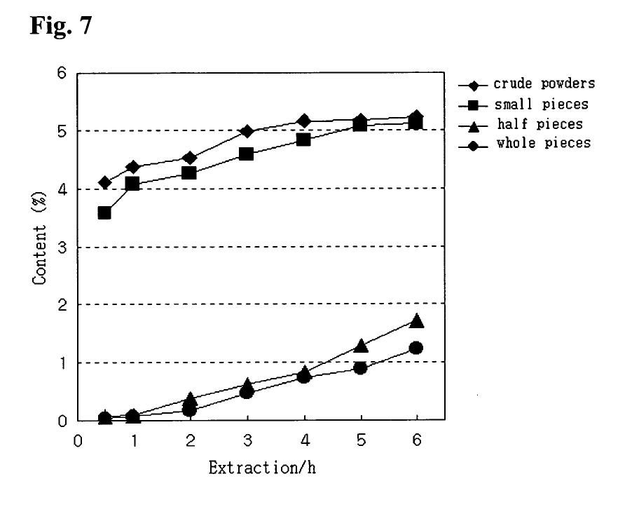

Therefore, we could find that extraction time and extract yield became improved as smaller of cutting sizes (FIG. 7).

### Experimental Example 8

### Amygdalin Extraction Yield Calibration of Husked Armenicae Semen Extract with Methanol

Each methanol extract of the crude powders, small pieces, half pieces and whole pieces from husked Armenicae Semen prepared in Example 6 was subjected to HPLC analysis according to a pretreatment of above described Example 6 and Experimental Example 1.

As a result of the experiment, the amygdalin content of husked Armenicae Semen was 5.5% from crude powders, 5.3% from small pieces, 4.0% from half pieces and 3.8% from whole pieces. The extraction time and yield of husked Armenicae Semen was similar to those of natural form Armenicae Semen according to cutting sizes, but extract yield was highly increased.

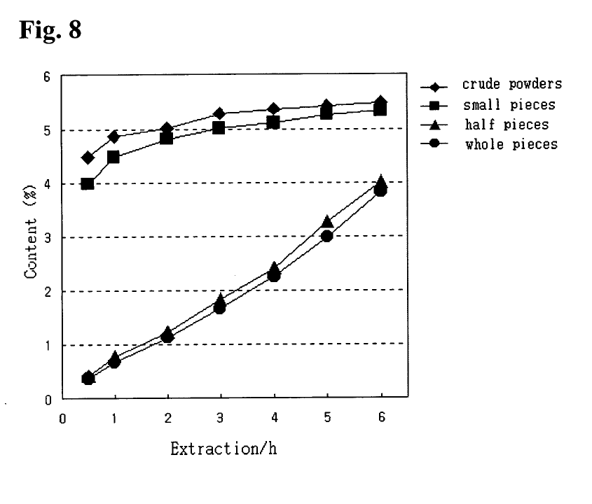

We could find that the extracted hours and extract yield were improved as smaller cutting sizes as above methanol extract of unhusked Armenicae Semen disclosed in Experimental Example 7 (FIG. 8).

### Experimental Example 9

### Amygdalin Extraction Yield Calibration of Unhusked Armenicae Semen Extract with Water

Each water extract of the crude powders, small pieces, half pieces and whole pieces from unhusked Armenicae Semen prepared in Example 7 was subjected to HPLC analysis according to a pre-treatment of above described Example 7 and Experimental Example 1.

As a result of the experiment, the amygdalin yield of unhusked Armenicae Semen was 0.5% from crude powders, 0.7% from small pieces, 1.2% from half pieces and 2.7% from whole pieces. And it took 6 hours to fulfill complete extraction.

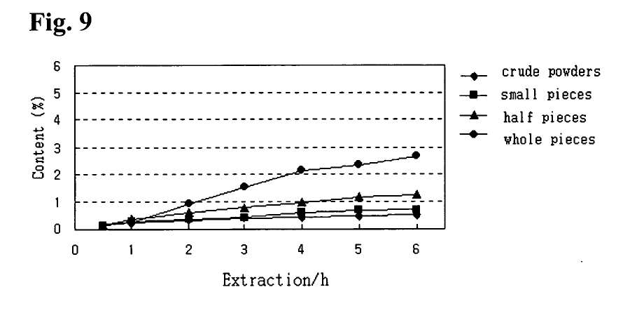

It was found that the amygdalin in the water extract from crude powders of Persicae Semen was mostly degraded by the effect of emulsin, a sort of hydrolase, but emulsin in methanol extract did not work. Actually amygdalin is decomposed into two molecules of glucose, a molecule of HCN and a molecule of benzaldehyde by emulsin, also β-glucosides such as salisin, arbutin, cellobiose and the like are also decomposed by emulsin. But it was confirmed that the effect of emulsin is decreased with the increase of the cutting size (FIG. 9).

### Experimental Example 10

### Amygdalin Extraction Yield Calibration of Husked Armenicae Semen Extract with Water

Each water extract of the crude powders, small pieces, half pieces and whole pieces from husked Armenicae Semen prepared in Example 8 was subjected to HPLC analysis according to a pre-treatment of above described Example 8 and Experimental Example 1.

As a result of the experiment, the amygdalin yield of husked Armenicae Semen was 1.9% from crude powders, 2.6% from small pieces, 4.7% from half pieces and 4.9% from whole pieces.

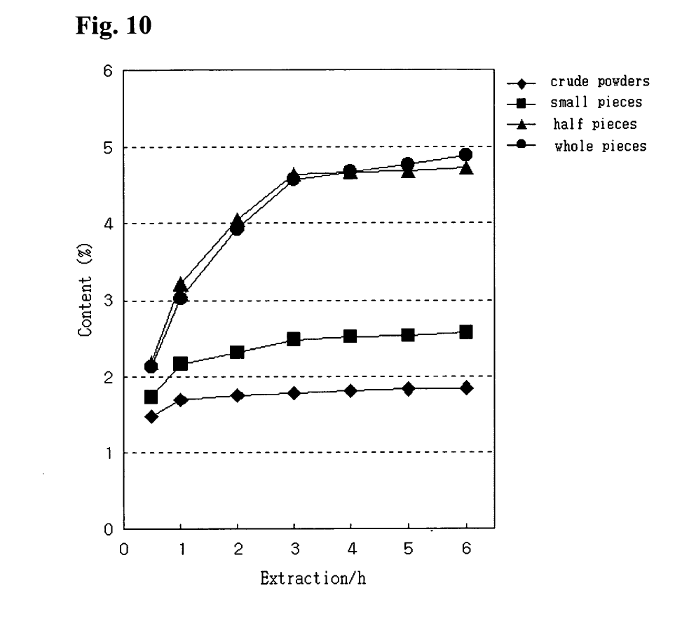

Extract of whole pieces of husked Armenicae Semen was less affected compared with other extracts, but the effect of emulsin was no completely removed in comparing with crude powders of methanol extract (FIG. 10).

### Experimental Example 11

### Amygdalin Extraction Yield Calibration of Husked Armenicae Semen Extract with Boiling Water

Each boiling water extract of the crude powders, small pieces, half pieces and whole pieces from husked Armenicae Semen prepared in Example 9 was subjected to HPLC analysis according to a pretreatment of above described Example 9 and Experimental Example 1.

As a result of experiment, the amygdalin yield of husked Armenicae Semen was 5.3% from crude powders, 5.3% from small pieces, 5.3% from half pieces and 5.5% from whole pieces.

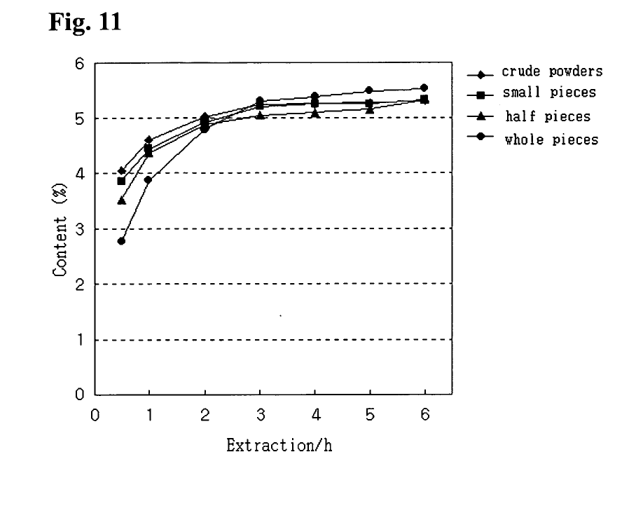

The amygdalin in crude powder extract was not affected by emulsin and the difference of surface area obtained by cutting form (FIG. 11).

### Experimental Example 12

Amygdalin extraction yield calibration of husked Armenicae Semen extract with boiling water comprising 0.1% citric acid

Each boiling water extract comprising 0.1% citric acid of the whole pieces from husked Armenicae Semen prepared in Example 10 was subjected to HPLC analysis according to a pretreatment of above described Example 10 and Experimental Example 1.

As a result of the experiment, the amygdalin yield of husked Armenicae Semen was 5.8% from whole pieces. We found out that this method showed remarkable extract yield more than of other methods and effective extract condition was also not converting amygdalin into neoamygdalin.

As shown in above described results, we found out the effective condition, which inhibited the conversion of amygdalin contained in Persicae Semen or Armenicae Semen into neoamygdalin and also confirmed that the inventive preparation method was affected by emulsin through controlling surface area of cutting.

1. A method characterized in minimizing the excised surface of Persicae Semen or Armenicae Semen in extracting Persicae Semen or Armenicae Semen with extraction solvent following acid pre-treatment.

2. The method according to claim 1 wherein said Persicae Semen or Armenicae Semen is used as unhusked.

3. The method according to claim 1 wherein said extraction solvent is selected at least one from the group consisting of water, methanol, butanol or the mixture thereof.

4. The method according to claim 3 wherein said water is extracted at the higher temperature than boiling point.

5. The method according to claim 1 wherein said acid pre-treatment is performed with the solvent containing at least one acid from the group consisting of citric acid, acetic acid, ascorbic acid or the mixture thereof.

6. The method according to claim 5 wherein said solvent containing acid is 0.05 to 0.5% of citric acid.

7. The method according to claim 1 wherein the method is characterized by using 0.1% citric acid-containing water solution with higher temperature than its boiling point.

8. (canceled)

9. (canceled)

10. (canceled)

11. The method according to claim 1 wherein the method is characterized by cutting Persicae Semen or Armenicae Semen in minimum for reducing contact areas between amygdalin and emulsin in the case of using water as an extraction solvent.

12. The method according to claim 2 wherein the method is characterized by cutting Persicae Semen or Armenicae Semen in minimum for reducing contact areas between amygdalin and emulsin in the case of using water as an extraction solvent.

13. The method according to claim 3 wherein the method is characterized by cutting Persicae Semen or Armenicae Semen in minimum for reducing contact areas between amygdalin and emulsin in the case of using water as an extraction solvent.

14. The method according to claim 4 wherein the method is characterized by cutting Persicae Semen or Armenicae Semen in minimum for reducing contact areas between amygdalin and emulsin in the case of using water as an extraction solvent.

15. The method according to claim 5 wherein the method is characterized by cutting Persicae Semen or Armenicae Semen in minimum for reducing contact areas between amygdalin and emulsin in the case of using water as an extraction solvent.

16. The method according to claim 6 wherein the method is characterized by cutting Persicae Semen or Armenicae Semen in minimum for reducing contact areas between amygdalin and emulsin in the case of using water as an extraction solvent.

17. The method according to claim 7 wherein the method is characterized by cutting Persicae Semen or Armenicae Semen in minimum for reducing contact areas between amygdalin and emulsin in the case of using water as an extraction solvent.

18. The method according to claim 11 wherein the method is characterized by cutting Persicae Semen or Armenicae Semen into the size smaller than half.

19. The method according to claim 12 wherein the method is characterized by cutting Persicae Semen or Armenicae Semen into the size smaller than half.

20. The method according to claim 13 wherein the method is characterized by cutting Persicae Semen or Armenicae Semen into the size smaller than half.

21. The method according to claim 14 wherein the method is characterized by cutting Persicae Semen or Armenicae Semen into the size smaller than half.

22. The method according to claim 15 wherein the method is characterized by cutting Persicae Semen or Armenicae Semen into the size smaller than half.

23. The method according to claim 16 wherein the method is characterized by cutting Persicae Semen or Armenicae Semen into the size smaller than half.

24. The method according to claim 17 wherein the method is characterized by cutting Persicae Semen or Armenicae Semen into the size smaller than half.

25. The method according to claim 1 wherein the method is characterized by using crude powders for providing with maximum surface of Persicae Semen or Armenicae Semen in the case of using methanol as an extraction solvent.

26. The method according to claim 2 wherein the method is characterized by using crude powders for providing with maximum surface of Persicae Semen or Armenicae Semen in the case of using methanol as an extraction solvent.

27. The method according to claim 3 wherein the method is characterized by using crude powders for providing with maximum surface of Persicae Semen or Armenicae Semen in the case of using methanol as an extraction solvent.
